(19) United States
(12) Patent Application Publication
(10) Pub. No.: US 2004/0131638 A1
(43) Pub. Date: Jul. 8, 2004

(54) Novel composition

(76) Inventors: Serge Debrus, Rixensart (BE); Nathalie Louise Mathy, Rixensart (BE); Gerald Voss, Rixensart (BE)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/475,784

(22) PCT Filed: Apr. 25, 2002
(86) PCT No.: PCT/EP02/04966

(30) Foreign Application Priority Data
Apr. 27, 2001 (GB) 0110431.4

(51) Int. Cl. A61K 39/295

(52) U.S. Cl. 424/202.1

(57) ABSTRACT

The present invention relates to a vaccine composition comprising at least one human immunodeficiency virus (HIV) antigen and either one or both of: i) at least one herpes simplex virus (HSV) antigen and ii) at least one human papillomavirus (HPV) antigen.

This invention relates to novel vaccine formulations, methods for preparing them and their use in prophylaxis and therapy. In particular the present invention relates to combination vaccines for administration to patients at risk of HIV infection. HIV-1 and HIV-2 are the causes of the acquired immune deficiency syndrome (AIDS) which is regarded as one of the world's major health problems. Although extensive research throughout the world has been conducted to produce a vaccine, such efforts thus far have not been successful.

The HIV envelope glycoprotein gp120 is the viral protein that is used for attachment to the host cell. This attachment is mediated by the binding to two surface molecules of helper T cells and macrophages, known as CD4 and one of the two chemokine receptors CCR-4 or CXCR-5. The gp120 protein is first expressed as a larger precursor molecule (gp160), which is then cleaved post-translationally to yield gp120 and gp41. The gp120 protein is retained on the surface of the virion by linkage to the gp41 molecule, which is inserted into the viral membrane.

The gp120 protein is the principal target of neutralising antibodies, but unfortunately the most immunogenic regions of the proteins (V3 loop) are also the most variable parts of the protein. Therefore, the use of gp120 (or its precursor gp160) alone as a vaccine antigen to elicit neutralising antibodies is thought to be of limited use for a broadly protective vaccine. The gp120 protein does also contain epitopes that are recognised by cytotoxic T lymphocytes (CTL). These effector cells are able to eliminate virus-infected cells, and therefore constitute a second major antiviral immune mechanism. In contrast to the target regions of neutralising antibodies some CTL epitopes appear to be relatively conserved among different HIV strains. For this reason gp120 and gp160 are considered to be useful antigenic components in vaccines that aim at eliciting cell-mediated immune responses (particularly CTL).

Non-envelope proteins of HIV-1 have been described and include for example internal structural proteins such as the products of the gag and pol genes and, other non-structural proteins such as Rev, Nef, Vif and Tat (Greene et al., New England J. Med, 324, 5, 308 et seq (1991) and Bryant et al. (Ed. Pizzo), Pediatr. Infect. Dis. J., 11, 5, 390 et seq (1992)).

The HIV gag gene encodes a precursor protein p55, which can assemble spontaneously into immature virus-like particles (VLPs). The precursor is then proteolytically cleaved into the major structural proteins p24 (capsid) and p18 (matrix), and into several smaller proteins.

HIV Tat and Nef are early proteins, that is, they are expressed early in infection and in the absence of structural protein.

HSV-2 is the primary etiological agent of herpes genitalis. HSV-1 is the causative agent of herpes labialis. Together, these viruses are characterised by their ability to induce both acute diseases and to establish a latent infection, primarily in neuronal ganglia cells.

Genital herpes is estimated to occur in about 5 million people in the U.S.A. alone with 500,000 clinical cases recorded every year (primary and recurrent infection). Primary infection typically occurs after puberty and is characterised by the localised appearance of painful skin lesions, which persist for a period of between 2 to 3 weeks. Within the following six months after primary infection 50% of patients will experience a recurrence of the disease. About 25% of patients may experience between 10-15 recurrent episodes of the disease each year. In immunocompromised patients the incidence of high frequency recurrence is statistically higher than in the normal patient population.

Both HSV-1 and HSV-2 virus have a number of glycoprotein components located on the surface of the virus. These are known as gB, gC, gD and gE etc.

Glycoprotein D is located on the viral membrane, and is also found in the cytoplasm of infected cells (Eisenberg R. J. et al; J of Virol 1980 35 428-435). It comprises 393 amino acids including a signal peptide and has a molecular weight of approximately 60 kD. Of all the HSV envelope glycoproteins this is probably the best characterised (Cohen et al J. Virology 60 157-166). In vivo it is known to play a central role in viral attachment to cell membranes. Moreover, glycoprotein D has been shown to be able to elicit neutralising antibodies in vivo (Eing et al J. Med. Virology 127: 59-65). However, latent HSV-2 virus can still be reactivated and induce recurrence of the disease despite the presence of high neutralising antibodies titre in the patients sera.

Papillomaviruses are small DNA tumour viruses, which are highly species specific. So far, over 70 individual human papillomavirus (HPV) genotypes have been described. HPVs are generally specific either for the skin (e.g. HPV-1 and -2) or mucosal surfaces (e.g. HPV-6 and -11) and usually cause benign tumours (warts) that persist for several months or years. Such benign tumours may be distressing for the individuals concerned but tend not to be life threatening, with a few exceptions.

Some HPVs are also associated with cancers. The strongest positive association between an HPV and human cancer is that which exists between HPV-16 and HPV-18 and cervical carcinoma. Cervical cancer is the most common malignancy in developing countries, with about 500,000 new cases occurring in the world each year. It is now technically feasible to actively combat primary HPV-16 infections, and even established HPV-16-containing cancers, using vaccines. For a review on the prospects for prophylactic and therapeutic vaccination against HPV-16 see Cason J., Clin. Immunother. 1994; 1(4) 293-306 and Hagenesee M. E., Infections in Medicine 1997 14(7) 555-556,559-564.

Other HPVs of particular interest are serotypes 31,33 and 45.

Today, the different types of HPVs have been isolated and characterised with the help of cloning systems in bacteria and more recently by PCR amplification. The molecular organisation of the HPV genomes has been defined on a comparative basis with that of the well-characterised bovine papillomavirus type 1 (BPV1).

Although minor variations do occur, all BPVs genomes described have at least seven early genes, E1 to E7 and two late genes L1 and L2. In addition, an upstream regulatory region harbors the regulatory sequences which appear to control most transcriptional events of the HPV genome.

E1 and E2 genes are involved in viral replication and transcriptional control, respectively and tend to be disrupted by viral integration. E6 and E7, and recent evidence implicate also E5 are involved in viral transformation.

In the HPVs involved in cervical carcinoma such as HPV 16 and 18, the oncogenic process starts after integration of viral DNA. The integration results in the inactivation of genes coding for the capsid proteins L1 and L2 and in installing continuous over expression of the two early proteins E6 and E7 that will lead to gradual loss of the normal cellular differentiation and the development of the carcinoma.

Carcinoma of the cervix is common in women and develops through a pre-cancerous intermediate stage to the invasive carcinoma which frequently leads to death. The intermediate stages of the disease is known as cervical intraepithelial neoplasia and is graded I to III in terms of increasing severity.

Clinically, HPV infection of the female anogenital tract manifests as cervical flat condylomas, the hallmark of which is the koilocytosis affecting predominantly the superficial and intermediate cells of the cervical squamous epithelium.

Koilocytes which are the consequence of a cytopathic effect of the virus, appear as multinucleated cells with a perinuclear clear halo. The epithelium is thickened with abnormal keratinisation responsible for the warty appearance of the lesion.

Such flat condylomas when positive for the HPV 16 or 18 serotypes, are high-risk factors for the evolution toward cervical intraepithelial neoplasia (CIN) and carcinoma in situ (CIS) which are themselves regarded as precursor lesions of invasive cervix carcinoma.

WO 96/19496 discloses variants of human papilloma virus E6 and E7 proteins, particularly fusion proteins of E6/E7 with a deletion in both the E6 and E7 proteins. These deletion fusion proteins are said to be immunogenic.

HPV L1 based vaccines are disclosed in WO94/00152, WO94/20137, WO93/02184 and WO94/05792. Such a vaccine can comprise the L1 antigen as a monomer, a capsomer or a virus like particle. Such particles may additionally comprise L2 proteins. L2 based vaccines are described for example in WO93/00436. Other HPV vaccines are based on the Early proteins, such as E7 or fusion proteins such as L2-E7.

The transmission of HIV is enhanced through genital lesions caused by other sexually transmitted pathogens (Fleming, D T, Wasserheit, J. N. From epidemiological synergy to public health policy and practice: the contribution of other sexually transmitted diseases to sexual transmission of HIV infection. Sex. Transm. Infect. 1999; 75:3-17). Major causes of genital lesions are Herpes Simplex Virus (HSV) and human papillomavirus (HPV). For example, HSV-2 infection is diagnosed frequently in African countries where HIV is also highly prevalent. An epidemiological survey in the Central African Republic revealed that there is a significant association between HSV and HIV (Mbopi-Kéou, F.-X., Grésenguet, G., Mayaud, P., Weiss, H. A., Gopal, R., Matta, M., Paul, J.-L., Brown, D. W. G., Hayes, R. J., Mabey, D. C. W., Bélec, L. Interactions between Herpes Simplex Virus type 2 and human Immunodeficiency Virus type 1 infection in African women: opportunities for intervention. J. Infect. Dis. 2000; 182:1090-1096). HSV-2 antibodies, virus shedding and HSV-2 DNA were present at a significantly higher rate in HIV-1 seropositive women. Furthermore, there was a correlation between the presence of HSV-2 DNA and HIV-1 RNA. These findings exemplify the interactions between the two pathogens in areas of high transmission of HIV.

There is still a need for the effective treatment and prevention of HIV. The present invention addresses this need.

In a first aspect the present invention provides a vaccine composition comprising:

(a) at least one human immunodeficiency virus (HIV) antigen; and either one or both of:

(b) at least one herpes simplex virus (HSV) antigen and

(c) at least one or several human papillomavirus (HPV) antigens

The present invention essentially provides for effective combination vaccines against both HIV and HSV and/or HPV. We demonstrate that simultaneous or co-administration of antigens from these viruses provokes an immune response against all antigens. Immunisation against both HIV and HSV and/or HPV can result in better protection from HIV infection (and vice versa). Even a partially effective prophylactic vaccine against HIV can be significantly enhanced by the addition or concomitant administration of a prophylactic or therapeutic HSV or HPV vaccine, for example.

The present invention further provides for the simultaneous administration of an HIV vaccine with an HSV vaccine and/or an HPV vaccine. Simultaneous administration is preferably achieved by admixture of appropriate antigens before vaccine delivery.

The invention also relates to the concomitant delivery of at least one HIV antigen with at least one herpes simplex virus (HSV) antigen and/or at least one human papillomavirus (HPV) antigen. Concomitant delivery relates to substantially simultaneous administration or co-administration of such antigen combinations. Co-administration may be at the same administration site or, more preferably, at different administration sites.

The vaccine composition of the invention thus includes both mixed antigen preparations and combinations of antigens for co-administration, for example in the form of a kit.

The administration of multiple vaccine antigens in the same vaccine formulation or concomitantly in separate formulations can lead to interference in the induction of immune responses to the single vaccine antigens (Schmitt et al. Primary vaccination of infants with diphtheria-tetanus-acellular pertassis-hepatitis B virus-inactivated polio virus and Haemophilus influenzae type b vaccines given as either separate or mixed injections. J. Pediatr. 2000, 137:304-312). It has been found that certain vaccine compositions according to the invention show no interference, that is to say that the immune response to each antigen in the composition of the invention is essentially the same as that which is obtained by each antigen given individually.

In a preferred aspect of the invention, the administration of multiple vaccine antigens of the invention in the same vaccine formulation or concomitantly in separate formulations has substantially no effect on the immunogenicity of the individual antigen components.

The invention also extends to compositions for which the immune response to an antigen or antigens from one viral component of the combination vaccine (e.g. an antigen from the HPV component) is reduced in comparison to the response generated by administration of that viral component in the absence of antigens from other viral components, provided that the antigen(s) or viral component is still capable of generating an immune response, preferably a protective immune response.

Preferably the combined vaccine has enhanced activity or effectiveness in respect of one or more of the diseases (HIV, HSV or HPV), when compared to the individual vaccine component alone.

In a preferred embodiment the HSV and/or HPV component of the vaccine is sufficiently immunogenic to reduce the number and/or severity and/or transmission effect of lesions which are involved in HIV transmission.

The invention also extends to a kit comprising:

(a) at least one human immunodeficiency virus (HIV) antigen; and either one or both of

(b) at least one herpes simplex virus (HSV) antigen and

(c) at least one human papillomavirus (HPV) antigen.

The kit suitably provides individual or combined vaccine combinations which can be used in the present invention to provide the necessary protection or treatment against HIV and/or HSV and/or HPV infection or disease.

The vaccine composition of the invention is of great benefit for administration to people who may be particularly at risk of HIV and/or HSV and/or HPV infection. Subjects who are already infected by HSV or HPV, for example, may also benefit from the combination vaccine as, in those subjects, immunisation may also be performed to decrease transmission of these viruses to their seronegative sexual partner, thereby protecting the partner against infection. The invention thus relates to a method of decreasing or preventing viral transmission, such as HIV viral transmission, comprising treatment with a vaccine of the present invention.

The vaccine of the invention is suitable for use in prevention or treatment of infection and/or disease.

Preferably, the vaccine combination of the present invention also comprises an adjuvant.

In one embodiment, the adjuvant of the present invention is a preferential stimulator of a TH1 cell response, also herein called a TH1 type response.

An immune response may be broadly divided into two extreme categories, being a humoral or cell mediated immune response (traditionally characterised by antibody and cellular effector mechanisms of protection respectively). These categories of response have been termed TH1-type responses (cell-mediated response), and TH2-type immune responses (humoral response).

Extreme TH1-type immune responses may be characterised by the generation of antigen specific, haplotype restricted cytotoxic T lymphocytes, and natural killer cell responses. In mice TH1-type responses are often characterised by the generation of antibodies of the IgG2a subtype, whilst in the human these correspond to IgG1 type antibodies. TH2-type immune responses are characterised by the generation of a range of immunoglobulin isotypes including in mice IgG1.

It can be considered that the driving force behind the development of these two types of immune responses are cytokines. High levels of TH1-type cytokines tend to favour the induction of cell mediated immune responses to the given antigen, whilst high levels of TH2-type cytokines tend to favour the induction of humoral immune responses to the antigen.

The distinction of TH1 and THI2-type immune responses is not absolute. In reality an individual will support an immune response which is described as being predominantly TH1 or predominantly TH2. However, it is often convenient to consider the families of cytokines in terms of that described in murine CD4+ve T cell clones by Mosmann and Coffman (Mosmann, T. R. and Coffman, R. L. (1989) TH1 and TH2 cells: different patterns of lymphokine secretion lead to differentf functional properties. Annual Review of Immunology, 7, p145-173). Traditionally, TH1-type responses are associated with the production of the INF-γ cytokines by T-lymphocytes. Other cytokines often directly associated with the induction of TH1-type immune responses are not produced by T-cells, such as IL-12. In contrast, TH2-type responses are associated with the secretion of IL-4, IL-5, IL-6, IL-10 and tumour necrosis factor-β(TNF-β).

It is known that certain vaccine adjuvants are particularly suited to the stimulation of either TH1 or TH2-type cytokine responses. Traditionally the best indicators of the TH1:TH2 balance of the immune response after a vaccination or infection includes direct measurement of the production of TH1 or TH2 cytokines by T lymphocytes in vitro after restimulation with antigen, and/or the measurement (at least in mice) of the IgG1:IgG2a ratio of antigen specific antibody responses.

Thus, a TH1-type adjuvant is one which stimulates isolated T-cell populations to produce high levels of TH1-type cytokines when re-stimulated with antigen in vitro, and induces antigen specific immunoglobulin responses associated with TH1-type isotype.

Adjuvants which are capable of preferential stimulation of the TH1 cell response are described in International Patent Application No. WO 94/00153 and WO 95/17209.

3 De-O-acylated monophosphoryl lipid A (3D-MPL) is one such adjuvant. This is known from GB 2220211 (Ribi). Chemically it is a mixture of 3 De-O-acylated monophosphoryl lipid A with 4, 5 or 6 acylated chains and is manufactured by Ribi Immunochem, Mont. A preferred form of 3 De-O-acylated monophosphoryl lipid A is disclosed in European Patent 0 689 454 B1 (SmithKline Beecham Biologicals SA). Other detoxified bacterial LPS molecules such as MPL can be also be used, and reference herein to 3D-MPL is taken also to cover such detoxified LPS molecules where appropriate. Other purified and synthetic lipopolysaccharides have been described (U.S. Pat. No. 6,005,099 and EP 0 729 473 B1; Hilgers et al., 1986, Int.Arch.Allergy.Immunol., 79(4):392-6; Hilgers et al., 1987, Immunology, 60(l):141-6; and EP 0 549 074 B1).

Preferably, the particles of 3D-MPL are small enough to be sterile filtered through a 0.22micron membrane (as described in European Patent number 0 689 454). 3D-MPL will be present in the range of 10 μg -100 μg preferably 25-50 μg per dose wherein the antigen will typically be present in a range 2-50 μg per dose.

A preferred form of 3D-MPL is in the form of an emulsion having a small particle size less than 0.2 μm in diameter, and its method of manufacture is disclosed in WO 94/21292. Aqueous formulations comprising monophosphoryl lipid A and a surfactant have been described in WO9843670A2.

The bacterial lipopolysaccharide derived adjuvants to be formulated in the adjuvant combinations of the present invention may be purified and processed from bacterial sources, or alternatively they may be synthetic. For example, purified monophosphoryl lipid A is described in Ribi (supra), and 3-O-Deacylated monophosphoryl or diphosphoryl lipid A derived from Salmonella sp. is described in GB 2220211 and U.S. Pat. No. 4,912,094. Particularly preferred bacterial lipopolysaccharide adjuvants are 3D-MPL and the P(1-6) glucosamine disaccharides described in U.S. Pat. No. 6,005,099 and EP 0 729 473 B1.

Accordingly, the LPS derivatives that may be used in the present invention are those immunostimulants that are similar in structure to that of LPS or MPL or 3D-MPL. In another aspect of the present invention the LPS derivatives may be an acylated monosaccharide, which is a sub-portion to the above structure of MPL.

A preferred derivative of LPS is a purified or synthetic lipid A of the following formula:

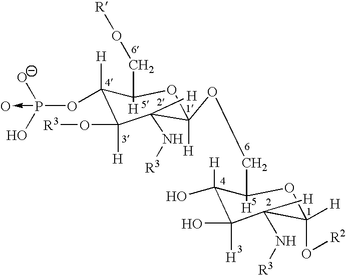

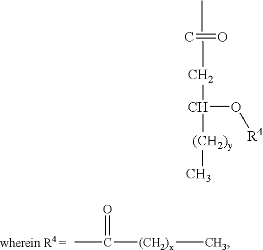

and wherein X and Y have a value of from 0 up to about 20.

Saponins are taught in: Lacaille-Dubois, M and Wagner H. (1996. A review of the biological and pharmacological activities of saponins. Phytomedicine vol 2 pp 363-386). Saponins are steroid or triterpene glycosides widely distributed in the plant and marine animal kingdoms. Saponins are noted for forming colloidal solutions in water which foam on shaking, and for precipitating cholesterol. When saponins are near cell membranes they create pore-like structures in the membrane which cause the membrane to burst. Haemolysis of erythrocytes is an example of this phenomenon, which is a property of certain, but not all, saponins.

Saponins are known as adjuvants in vaccines for systemic administration. The adjuvant and haemolytic activity of individual saponins has been extensively studied in the art (Lacaille-Dubois and Wagner, supra). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., CritRev TherDrug Carrier Syst, 1996, 12 (1-2):1-55; and EP 0 362 279 B1. Particulate structures, termed Immune Stimulating Complexes (ISCOMS), comprising fractions of Quil A are haemolytic and have been used in the manufacture of vaccines (Morein, B., EP 0 109 942 B1; WO 96/11711; WO 96/33739). The haemolytic saponins QS21 and QS 17 (HPLC purified fractions of Quil A) have been described as potent systemic adjuvants, and the method of their production is disclosed in U.S. Pat. No. 5,057,540 and EP 0 362 279 B1. Other saponins which have been used in systemic vaccination studies include those derived from other plant species such as Gypsophila and Saponaria (Bomford et al., Vaccine, 10(9):572-577, 1992).

Another preferred adjuvant comprises a saponin, for example as described above.

A preferred adjuvant comprises QS21, an Hplc purified non-toxic fraction derived from the bark of Quillaja Saponaria Molina. Optionally this may be admixed with 3 De-O-acylated monophosphoryl lipid A (3D-MPL), optionally together with an carrier.

Non-reactogenic adjuvant formulations containing QS21 have been described previously (WO 96/33739). Such formulations comprising QS21 and cholesterol have been shown to be successful TH1 stimulating adjuvants when formulated together with an antigen. Thus vaccine compositions which form part of the present invention may include a combination of QS21 and cholesterol.

Further adjuvants which are preferential stimulators of TH1 cell response include immunomodulatory oligonucleotides, for example unmethylated CpG sequences as disclosed in WO 96/02555.

CpG when formulated into vaccines, is generally administered in free solution together with free antigen (WO 96/02555; McCluskie and Davis, supra) or covalently conjugated to an antigen (WO 98/16247), or formulated with a carrier such as aluminium hydroxide ((Hepatitis surface antigen) Davis et al. supra ; Brazolot-Millan et al., Proc.Natl.Acad.Sci., USA, 1998, 95(26), 15553-8). Other preferred adjuvant combinations comprise CpG and a saponin.

Combinations of different TH1 stimulating adjuvants, such as those mentioned hereinabove, are also contemplated as providing an adjuvant which is a preferential stimulator of TH1 cell response. For example, QS21 can be formulated together with 3D-MPL. The ratio of QS21:3D-MPL will typically be in the order of 1:10 to 10:1; preferably 1:5 to 5:1 and often substantially 1:1. The preferred range for optimal synergy is 2.5:1 to 1:1 3D-MPL: QS21.

Preferably a carrier is also present in the vaccine composition according to the invention. The carrier may be an oil in water emulsion, or an aluminium salt, such as aluminium phosphate or aluminium hydroxide.

A preferred oil-in-water emulsion comprises a metabolisible oil, such as squalene, alpha tocopherol and Tween 80. In a particularly preferred aspect the antigens in the vaccine composition according to the invention are combined with QS21 and 3D-MPL in such an emulsion. Additionally the oil in water emulsion may contain span 85 and/or lecithin and/or tricaprylin.

In a particularly preferred aspect the antigens in the vaccine composition according to the invention are combined with 3D-MPL and alum.

Typically for human administration QS21 and 3D-MPL will be present in a vaccine in the range of 1 μg-200 μg, such as 10-100 μg, preferably 10 μg-50 μg per dose. Typically the oil in water will comprise from 2 to 10% squalene, from 2 to 10% alpha tocopherol and from 0.3 to 3% tween 80. Preferably the ratio of squalene: alpha tocopherol is equal to or less than 1 as this provides a more stable emulsion. Span 85 may also be present at a level of 1%. In some cases it may be advantageous that the vaccines of the present invention will frrther contain a stabiliser.

Non-toxic oil in water emulsions preferably contain a non-toxic oil, e.g. squalane or squalene, an emulsifier, e.g. Tween 80, in an aqueous carrier. The aqueous carrier may be, for example, phosphate buffered saline.

A particularly potent adjuvant formulation involving QS21, 3D-MFL and tocopherol in an oil in water emulsion is described in WO 95/17210.

Preferred combinations of adjuvant and antigen comprise the HIV gp120 and Nef-Tat proteins in combination with QS21, 3D-MPL in an oil in water emulsion as described in WO 95/17210.

The optimisation of antigens with adjuvants for use in the present invention is within the realm of the person skilled in the art.

In another aspect of the invention, the vaccine may contain DNA encoding one or more of the HIV, HSV or HPV polypeptides of interest, such that the polypeptide is generated in situ. The DNA may be present within any of a variety of delivery systems known to those of ordinary skill in the art, including nucleic acid expression systems such as plasmid DNA, bacteria and viral expression systems. Numerous gene delivery techniques are well known in the art, such as those described by Rolland, Crit. Rev. Therap. Drug Carrier Systems 15:143-198, 1998 and references cited therein. Appropriate nucleic acid expression systems contain the necessary DNA sequences for expression in the patient (such as a suitable promoter and terminating signal). When the expression system is a recombinant live microorganism, such as a virus or bacterium, the gene of interest can be inserted into the genome of a live recombinant virus or bacterium. Inoculation and in vivo infection with this live vector will lead to in vivo expression of the antigen and induction of immune responses. Viruses and bacteria used for this purpose are for instance: poxviruses (e.g; vaccinia, fowlpox, canarypox, modified poxviruses e.g. Modified Virus Ankara (MVA)), alphaviruses (Sindbis virus, Semliki Forest Virus, Venezuelian Equine Encephalitis Virus), flaviviruses (yellow fever virus, Dengue virus, Japanese encephalitis virus), adenoviruses, adeno-associated virus, picomaviruses (poliovirus, rhinovirus), herpesviruses (varicella zoster virus, etc), Listeria, Salmonella, Shigella, Neisseria, BCG. These viruses and bacteria can be virulent, or attenuated in various ways in order to obtain live vaccines. Such live vaccines also form part of the invention.

Thus, the HIV, HSV or HPV components of a preferred vaccine according to the invention may be provided in the form of polynucleotides encoding the desired proteins. Polynucleotides may be in the form of vectors that encode single proteins, for example, or may be single vectors that express multiple antigens from one or more of the three pathogens.

Furthermore, immunisations according to the invention may be performed with a combination of protein and DNA-based formulations. Prime-boost immunisations are considered to be effective in inducing broad immune responses. Adjuvanted protein vaccines induce mainly antibodies and T helper immune responses, while delivery of DNA as a plasmid or a live vector induces strong cytotoxic T lymphocyte (CTL) responses. Thus, the combination of protein and DNA vaccination will provide for a wide variety of immune responses. This is particularly relevant in the context of HIV, since both neutralising antibodies and CTL are thought to be important for the immune defence against HIV.

In accordance with the invention a schedule for vaccination with HIV and either one or both of HSV and HPV antigens alone or in combination, may comprise the sequential (“prime-boost”) or simultaneous administration of protein antigens and DNA encoding the above-mentioned proteins. The DNA may be delivered as plasmid DNA or in the form of a recombinant live vector, e.g. a poxvirus vector or any other suitable live vector such as those described herein. Protein antigens may be injected once or several times followed by one or more DNA administrations, or DNA may be used first for one or more administrations followed by one or more protein immunisations.

In a fuirther embodiment of the invention a schedule for vaccination with HIV and either one or both of HSV and HPV antigens alone or in combination, may comprise the sequential (“prime-boost”) administration of DNA encoding the above-mentioned proteins in a combination of different DNA delivery modes. For example, naked DNA may be used first for one or more administrations followed by one or more DNA administrations in the form of a recombinant live vector.

The HIV antigens of the present invention preferably comprise a combination of an HIV envelope protein or derivative thereof with a regulatory or non-structural protein e.g. Gag, Pol, Rev, Nef, Vif or Tat.

The HIV antigen(s) in the composition of the present invention is preferably

(a) an HIV Nef protein or derivative thereof;

(b) an HWV Tat protein or derivative thereof;

(c) an HIV Nef protein or derivative thereof linked to an HIV Tat protein or derivative thereof;

(d) an HIV Env protein (gp160 or gp120) or derivative thereof;

(e) HIV Nef protein or derivative thereof linked to an HIV Tat protein or derivative thereof in combination with gp120 or derivative thereof;

(f) an HWV Gag or Pol protein or derivative thereof.

Most preferred is a nef-tat fusion in combination with gp120 as disclosed in WO 01/54719, the whole contents of which are incorporated herein by reference. Preferably the Tat, Nef or Nef-Tat act in synergy with gp120 in the treatment or prevention of HIV, most preferably there being synergy between nef-tat and gp120.

Derivatives encompassed within the present invention include molecules with a C-terminal Histidine tail which preferably comprises between 5-10 Histidine residues. Generally, a histidine tail containing n residues is represented herein as His (n). The presence of an histidine (or ‘His’) tail aids purification.

In a preferred embodiment some or all of the proteins are expressed with a Histidine tail comprising between 5 to 10 and preferably six Histidine residues. These are advantageous in aiding purification. Separate expression, in yeast (Saccharomyces cerevisiae), of Nef (Macreadie I.G. et al., 1993, Yeast 9 (6) 565-573) and Tat (Braddock M et al., 1989, Cell 58 (2) 269-79) has been reported. The expression of a fusion construct Nef-Tat-His is described in WO99/16884.

Derivatives encompassed within the present invention also include mutated proteins. The term ‘mutated’ is used herein to mean a molecule which has undergone deletion, addition or substitution of one or more amino acids using well known techniques for site directed mutagenesis or any other conventional method. This definition is not limited to HIV antigens and applies to all antigens for use in the vaccine of the present invention. Other suitable derivative forms include fusions proteins, cross-linked proteins, protein truncations and codon optimised sequences, including nucleotides encoding such derivatives.

Derivatives of an antigen are also preferably substantially as immunogenic as the original antigen, or encode an antigen which is substantially as immunogenic as the original antigen.

The HPV antigen in the composition of the invention is preferably derived from HPV 16 and/or 18, or from HPV 6 and/or 11, or HPV 31, 33, 45, 52, 58, 35, 56, and 59.

In one preferred embodiment the HPV antigen in the vaccine composition according to the invention comprises the major capsid protein L1 of HPV and optionally the L2 protein, particularly from HPV 16 and/or HPV 18. In this embodiment, the preferred form of the L1 protein is a truncated L1 protein, most preferably a C terminal truncation. Preferably the L1, optionally in a L1-L2 fusion, is in the form of a virus-like particle (VLP). Methods for the production of virus like particles are well known in the art. The L1 protein may be fused to another HPV protein, in particular E7 to form an L1-E7 fusion. Chimeric VLPs comprising L1-E or L1-L2-E are particularly preferred.

In another preferred embodiment, the HPV antigen in the composition of the invention is derived from an E6 or E7 protein, in particular E6 or E7 linked to an immunological fiusion partner having T cell epitopes.

In a preferred form of this embodiment of the invention, the immunological fusion partner is derived from protein D of Heamophilus influenza B. Preferably the protein D derivative comprises approximately the first ⅓ of the protein, in particular approximately the first N-terminal 100-110 amino acids.

Preferred fusion proteins in this embodiment of the invention comprise Protein D-E6 from HPV 16, Protein D-E7 from HPV 16 Protein D-E7 from HPV 18 and Protein D-E6 from HPV 18. The protein D part preferably comprises the first ⅓ of protein D.

In still another embodiment of the invention, the HPV antigen is in the form of an L2-E7 fusion, particularly from HPV 6 and/or HPV 11.

The HPV proteins of the present invention preferably are expressed in E. coli. In a preferred embodiment the proteins are expressed with a Histidine tail comprising between 5 to 9 and preferably six Histidine residues. These are advantageous in aiding purification. The description of the manufacture of such proteins is fully described in UK patent application number GB 9717953.5, published as Wb99/10375.

The HPV antigen in the vaccine composition may be adsorbed onto Al(OH)₃.

The HSV antigen in the composition of the invention is preferably derived from HSV-2, typically glycoprotein D. Glycoprotein D is located on the viral membrane, and is also found in the cytoplasm of infected cells (Eisenberg R. J. et al; J of Virol 1980, 35, 428-435). It comprises 393 amino acids including a signal peptide and has a molecular weight of approximately, 60 kD. Of all the HSV envelope glycoproteins this is probably the best characterised (Cohen et al; J. of Virology, 60, 157-166). In vivo it is known to play a central role in viral attachment to cell membranes. Moreover, glycoprotein D has been shown to be able to elicit neutralising antibodies in vivo (Eing et al J. Med. Virology 127:59-65). However, latent HSV-2 virus can still be reactivated and induce recurrence of the disease despite the presence of high neutralising antibodies titre in the patients sera.

In a preferred embodiment of the invention the HSV antigen is a truncated HSV-2 glycoprotein D of 308 amino acids which comprises amino acids 1 through 306 naturally occurring glycoprotein with the addition Asparagine and Glutamine at the C terminal end of the truncated protein devoid of its membrane anchor region. This form of the protein includes the signal peptide which is cleaved to yield a mature 283 amino acid protein. The production of such a protein in Chinese Hamster ovary cells has been described in Genentech's European patent EP-B-139 417.

The recombinant mature HSV-2 glycoprotein D truncate is preferably used in the vaccine formulations of the present invention and is designated rgD2t.

A combination of this HSV-2 antigen in combination with the adjuvant 3D-MPL has been described in WO 92/16231.

Most preferred is a vaccine comprising gp120 and a nef-tat fusion in combination with an HPV VLP comprising L1 (full length or truncated) and/or rgD2t from HSV.

The present invention in a fuirther aspect provides a vaccine formulation as herein described for use in medical therapy, particularly for use in the treatment or prophylaxis of HIV infection, human papillomavirus infections and herpes simplex virus infections.

The vaccine of the present invention will contain an immunoprotective quantity of the antigens and may be prepared by conventional techniques.

Vaccine preparation is generally described in Pharmaceutical Biotechnology, Vol. 61 Vaccine Design—the subunit and adjuvant approach, edited by Powell and Newman, Plenum Press, 1995. New Trends and Developments in Vaccines, edited by Voller et al., University Park Press, Baltimore, Md., U.S.A. 1978. Encapsulation within liposomes is described, for example, by Fullerton, U.S. Pat. No. 4,235,877. Conjugation of proteins to macromolecules is disclosed, for example, by Likhite, U.S. Pat. No. 4,372,945 and by Armor et al., U.S. Pat. No. 4,474,757.

The amount of protein in each vaccine dose is selected as an amount which induces an immunoprotective response without significant, adverse side effects in typical vaccinees. Such amount will vary depending upon which specific immunogen is employed. Generally, it is expected that each dose will comprise 1-1000 μg of protein, preferably 2-100 μg, most preferably 4-40 μg. An optimal amount for a particular vaccine can be ascertained by standard studies involving observation of antibody titres and other responses in subjects. Following an initial vaccination, subjects may receive one or several boosts in about 4 to 8 week intervals.

In addition to vaccination of persons susceptible to HIV and/or either one or both of HPV and/or HSV infections, the pharmaceutical compositions of the present invention may be used to treat, immunotherapeutically, patients suffering from the said viral infections.

Thus the present invention relates to a method of treatment comprising delivering to an individual in need of such treatment an effective amount of a vaccine against both HIV and HSV and/or BPV. The method is for the prevention or treatment of infection or disease caused by HIV and/or HPV and/or HSV, as appropriate.

In a further aspect of the present invention there is provided a method of manufacture for a vaccine as herein described, wherein the method comprises mixing a human immunodeficiency virus antigen with either one or both of a human papilloma virus antigen and a herpes simplex virus antigen. Alternatively manufacture may comprise mixing polynucleotides encoding suitable antigens, or combining polynucleotide and protein, to produce the vaccines of the invention. Preferably the antigens are formulated with an adjuvant such as a TH-1 inducing adjuvant, for example 3D-MPL and, preferably, a carrier, for example alum.

If desired, other antigens may be added, in any convenient order, to provide multivalent vaccine compositions as described herein.

The vaccine preparations of the present invention may be used to protect or treat a mammal susceptible to, or suffering from disease, by means of administering said vaccine via

(a) a mucosal route, such as the oral/bucal/intestinal/vaginal/rectal or nasal route;

(b) by parenteral delivery, for example intramuscular, or subcutaneous administration; or

(c) by transdermal, intradermal, intra-epithelial, topical or transcutaneous delivery.

The invention also relates to delivery devices comprising the vaccine of the invention, for example, devices adapted for intradermal or mucosal delivery or gene guns. Suitable delivery devices are well known in the art.

The vaccine preparations of the present invention may optionally be administered by a combination of the routes listed.

The present invention is illustrated by the following Examples which are illustrative but not limiting upon the present invention, wherein:

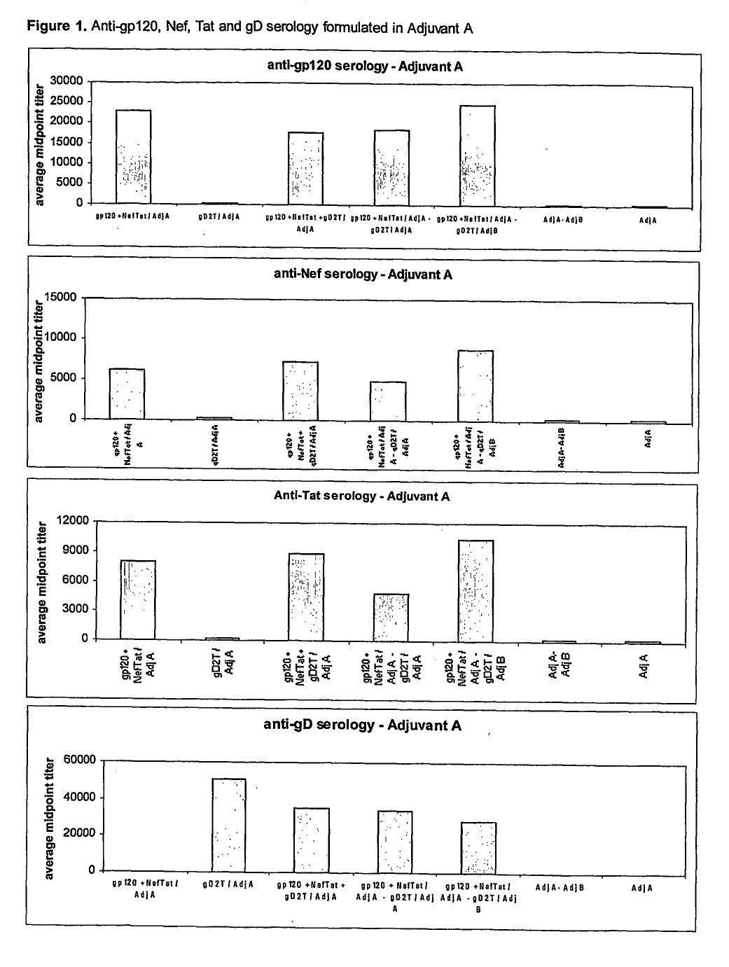

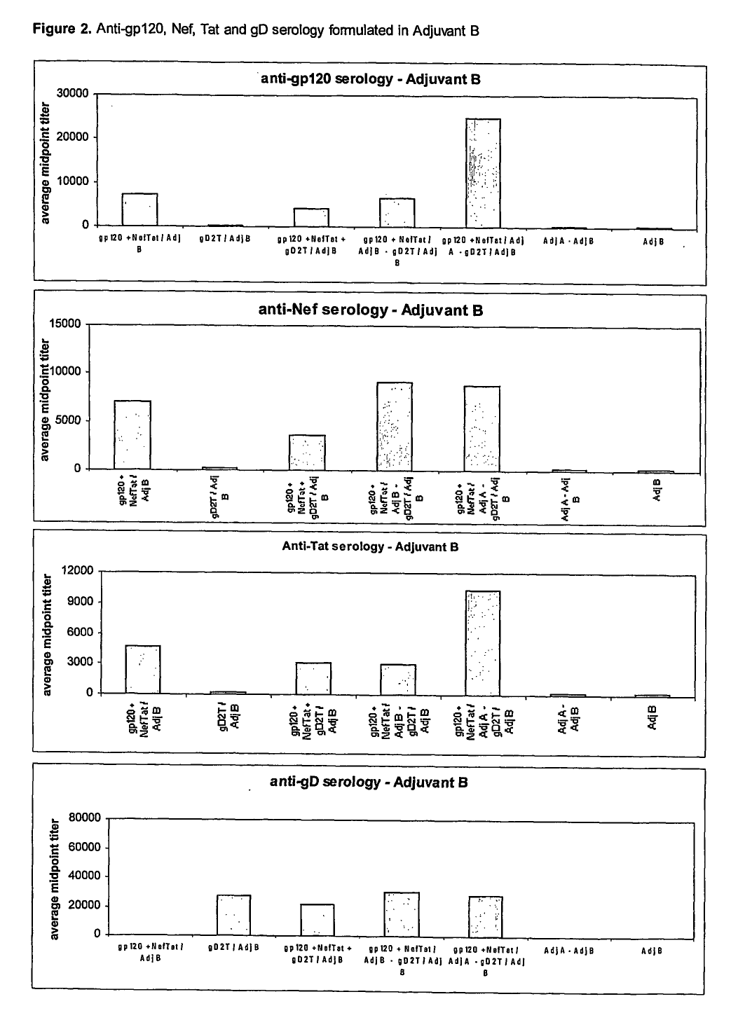

FIGS. 1 and 2 illustrates antibody responses to gp120, Nef, Tat and HSV gDt2 in different formulations of the present invention.

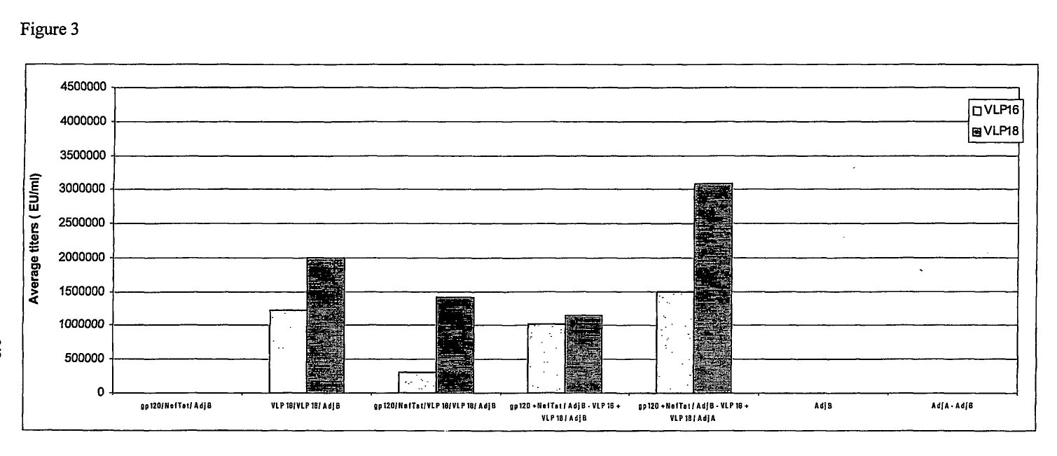

FIGS. 3 to 6 illustrate antibody responses to gp120, Nef, Tat and HPV in different formulations of the present invention.

### Example 1

### HIV/HSV Immunisations

Groups of 10 mice were immunised twice at two week intervals (days 0 & 14) with a combination of HIV antigens (gp120/nef-tat fision protein as described in WO/0 154719 incorporated herein by reference) and/or an HSV antigen gD2t (see for example WO 92/16231). 20 μg of gp120 and 4 μg of the nef-tat protein were used, with 4 μg of gD2t. The antigens were formulated in either of the adjuvants ‘A’ or ‘B’, ‘A’ being an oil in water emulsion containing QS21 and 3D MPL as described in the patent application WO95/17210 and ‘B’ being a combination of 3D MPL and an aluminium salt as described in patent application WO/0023105. Negative controls, with either or both adjuvants alone were also included. Two weeks following the booster immunisation (at day 28), the animals were sacrificed and sera collected for analysis of the immune response induced by these formulations.

TABLE 1
Experimental outline

| | IM immunisation leg 1 | | IM immunisation leg 2 | |
|---|---|---|---|---|
| Group | Antigens | Adjuvant | Antigens | Adjuvants |
| 1 | gp120/NefTat | A | — | — |
| 2 | gD2T | A | — | — |
| 3 | gp120/NefTat/gD2T | A | — | — |
| 4 | gp120/NefTat | A | gD2T | A |
| 5 | gp120/NefTat | A | gD2T | B |
| 6 | — | A | — | — |
| 7 | gp120/NefTat | B | — | — |
| 8 | gD2T | B | — | — |
| 9 | gp120/NefTat/gD2T | B | — | — |
| 10 | gp120/NefTat | B | gD2T | B |
| 11 | — | A | — | B |
| 12 | — | B | — | — |

### Antibody Response

Sera from the immunised mice from each group were analysed individually for gp120-, Nef-, Tat- and gD-specific antibody responses. Standard ELISA analysis was used, and such a method can be employed to assess suitability of antigens for use in the vaccine of the invention.

The results in FIGS. 1 and 2 show that both simultaneous delivery of HIV and HSV anitgens and concomitant delivery of HIV and HSV antigens (in different injection sites) generates an immune response to each component.

### EXAMPLE 2

### HIV/HPV Immunisations

The same general protocol used in Example 1 was employed to test the combinations of HIV and HPV. The gD component of HSV was replaced in these experiments by L1 VLPs from HPV 16 and HPV 18, 2 μg of each VLP.

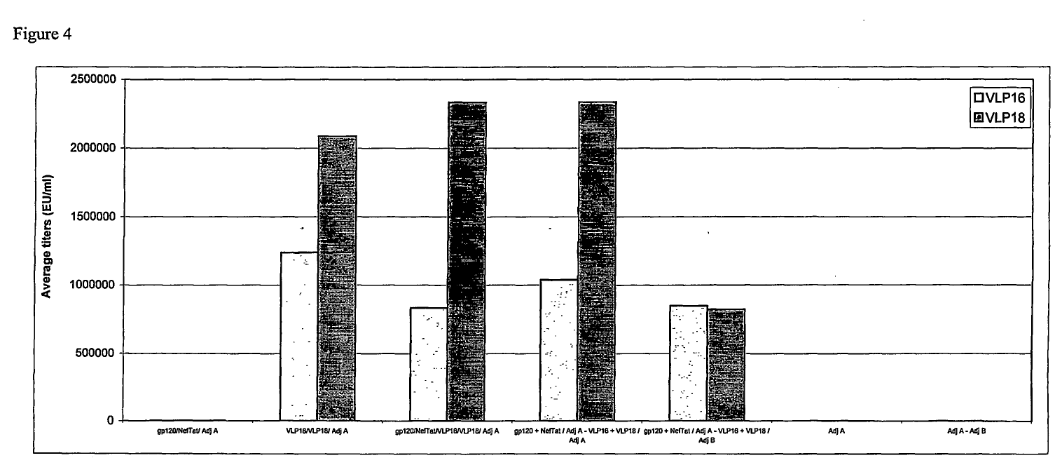

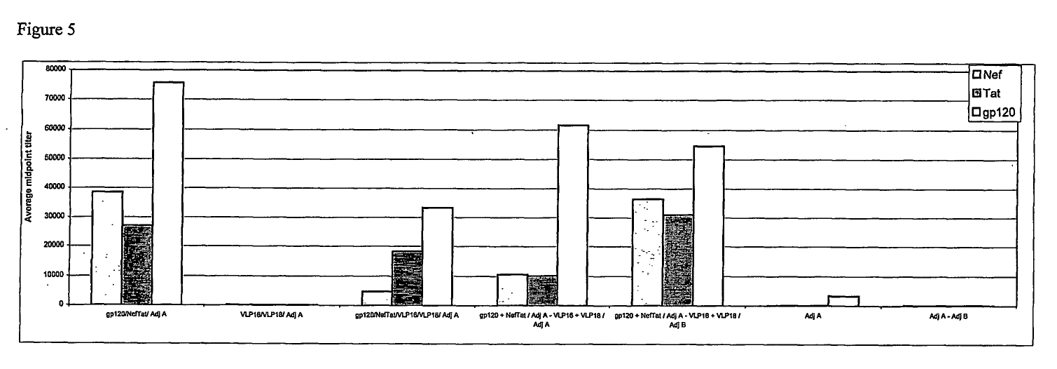

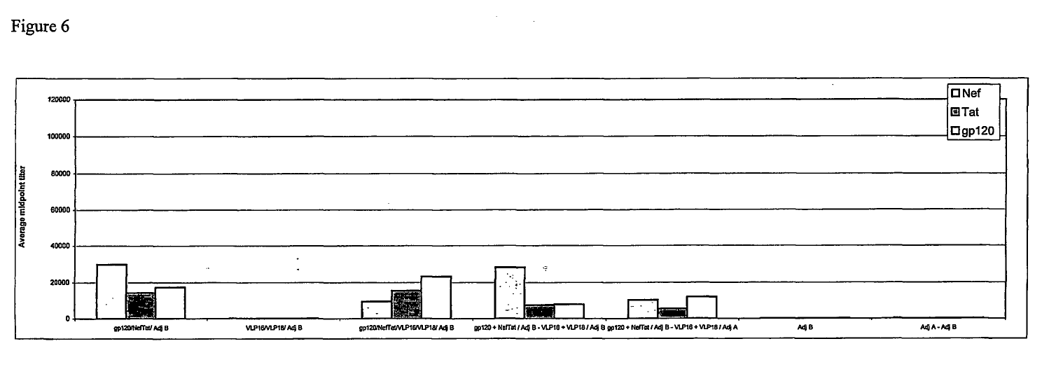

FIGS. 3 and 4 shows the average antibody titre generated against HPV 16 and 18 L1 VLPs. FIGS. 5 and 6 shows the average midpoint antibody titre generated against the HIV components Nef, tat and gp120.

The results in FIGS. 3-6 show that both simultaneous delivery of HIV and HPV antigens and concomitant delivery of HIV and HPV antigens (in different injection sites) generates an immune response to each component.

1. A vaccine composition comprising:
(a) at least one human immunodeficiency virus (HIV) antigen; and either one or both of
(b) at least one herpes simplex virus (HSV) antigen and
(c) at least one human papillomavirus (HPV) antigen.

2. A vaccine composition as claimed in claim 1 wherein the HIV antigen is selected from the group consisting of; gp160, gp120, nef, tat, a nef-tat or tat-nef fusion protein, gag, pol or immunologically active derivatives thereof.

3. A vaccine composition as claimed in claim 2 wherein the vaccine comprises HIV antigens gp120 and a nef-tat fusion protein.

4. A vaccine composition according to claim 2 or 3 wherein the Tat, Nef or Nef-tat act in synergy with gp120.

5. A vaccine composition according to any preceding claim wherein the HPV antigen is selected from the group consisting of L1, L2, E6 and E7 or combinations thereof, optionally in the form of a fusion protein or a truncate.

6. A vaccine composition as claimed in claim 5 wherein the HPV antigen is a virus like particle comprising the L1 protein or a C terminal truncation thereof.

7. A vaccine composition according to any preceding claim wherein the HSV antigen is HSV-2 gD or a truncate thereof

8. A vaccine composition as claimed in any one of the preceding claims which further comprises an adjuvant.

9. A vaccine composition according to claim 8 wherein the adjuvant is a preferential stimulator of TH1-cell response.

10. A vaccine composition according to claim 9 wherein the preferential stimulator of TH1-cell response is selected from the group of adjuvants comprising: 3D-MPL, 3D-MPL wherein the size of the particles of 3D-MPL is preferably about or less than 100 nm, QS21, a mixture of QS21 and cholesterol and a CpG oligonucleotide, or combinations thereof.

11. A composition according to claim 9 or 10 which additionally comprises an oil in water emulsion.

12. A vaccine composition according to claim 11 comprising HIV gp120 and a fusion protein of HIV Nef with HIV Tat in combination with QS21, 3D-MPL and an oil-in-water emulsion.

13. A vaccine composition according to any preceding claim wherein at least one antigen is in the form of DNA or a live vector.

14. A vaccination kit comprising:
(a) at least one human immunodeficiency virus (HIV) antigen; and either one or both of
(b) at least one herpes simplex virus (HSV) antigen; and
(c) at least one human papillomavirus (HPV) antigen.

15. A method of medical treatment comprising delivering to an individual in need of such treatment an effective amount of a vaccine against HIV and HSV and/or HPV.

16. A method according to claim 15, comprising the delivery of a vaccine against HIV and HSV.

17. A method according to claim 15, comprising the delivery of a vaccine against HIV and HPV.

18. A method according to any of claims 15 to 17 comprising delivery of a single vaccine containing a mixture of antigens from HIV and HSV and/or HPV.

19. A method according to any of claims 15 to 17 wherein vaccines against HIV and HSV and/or HPV are co-administered at separate administration sites.

20. Use of an BPV antigen in the preparation of a medicament for the prevention ot treatment of HIV or HSV infection or disease.

21. Use of an HSV antigen in the preparation of a medicament for the prevention or treatment of HIV or HPV infection or disease.

22. Use according to any of claims 20 or 21 wherein the use is for prevention or treatment of HIV infection or disease.

23. A method for the preparation of a vaccine according to any of claims 1-13 comprising combining at least one human inmmunodeficiency virus (HIV) antigen with either one or both of:
i) at least one herpes simplex virus (HSV) antigen; and
ii) at least one human papillomavirus (HPV) antigen.

24. A method of decreasing HIV viral transmission, the method comprising treatment with a vaccine according to any of claims 1-13.
